(19) United States
(12) Patent Application Publication
(10) Pub. No.: US 2025/0011724 A1
(43) Pub. Date: Jan. 9, 2025

(54) COMPOSITION COMPRISING DECELLULARIZED UTERINE TISSUE-DERIVED EXTRACELLULAR MATRIX AND USE THEREOF

(71) Applicant: Industry-Academic Cooperation Foundation, YONSEI UNIVERSITY, Seoul (KR)

(72) Inventors: Seung Woo CHO, Seoul (KR); Yoon Hee JIN, Seoul (KR); Yi Sun CHOI, Seoul (KR); Eun Ju PARK, Seoul (KR)

(21) Appl. No.: 18/708,373

(22) PCT Filed: Nov. 10, 2022
(86) PCT No.: PCT/KR2022/017662

§ 371 Date: May 8, 2024

(30) Foreign Application Priority Data
Nov. 10, 2021 (KR) 10-2021-0153961

(51) Int. Cl. C12N 5/071; C12N 5/00

(52) U.S. Cl. CPC C12N 5/0682; C12N 5/0062; C12N 5/0068; C12N 2500/05; C12N 2501/415; C12N 2501/998; C12N 2513/00

(57) ABSTRACT

A composition comprising a decellularized uterine tissue-derived extracellular matrix and a use thereof and, more specifically, a hydrogel composition for three-dimensional culturing an endometrial organoid, a preparation method therefor, and a use thereof are described. The hydrogel composition comprising a decellularized uterine tissue-derived extracellular matrix can be utilized to fabricate an endometrial organoid highly similar to in vivo uterine tissues and organs through the properties, components, and physical properties of the decellularized uterine tissue-derived extracellular matrix, and such endometrial organoids can be advantageously used for in-vivo implantation and in drug tests for various uterus-related diseases.

### TECHNICAL FIELD

The present invention relates to a composition comprising decellularized uterine tissue-derived extracellular matrix and the use thereof, and specifically, to a hydrogel composition for three-dimensional culture of endometrial organoids, endometrial organoids, preparation methods therefor, and the uses thereof.

### BACKGROUND ART

Organoids, which have recently attracted attention, are tissue analogs that may be used in various clinical applications, including new drug screening, drug toxicity evaluation, disease modeling, cell therapy products, and tissue engineering, and are a technology that is rapidly growing worldwide. Organoids are three-dimensional structures that not only consist of multiple cell types that make up specific organs and tissues of the human body, but can also mimic complex interactions between the cell types. Therefore, organoids can be applied as a much more accurate in vitro model platform than previously mainly used drug evaluation models such as simple cell line models or animal models.

Numerous research teams around the world studying these organoids are commonly using Matrigel products as culture scaffolds to culture organoids. However, since Matrigel is a material extracted from mouse sarcoma tissue, it is difficult to maintain the quality of the product uniformly, and Matrigel is expensive and has safety problems such as infection and transmission of animal pathogens and viruses. Therefore, Matrigel as an organoid culture system has a lot of problems to be solved. In particular, as a material derived from cancer tissue, Matrigel cannot provide an optimal tissue-specific microenvironment necessary for culturing a specific tissue organoid. Although there have been some studies on the development of polymer-based hydrogels to replace Matrigel, a material that can replace Matrigel has not yet been reported.

Meanwhile, the uterus is a female reproductive organ where a fertilized egg implants and develops into a fetus. Also, the uterus is a very important female organ where a fertilized egg implants, the placenta attaches, and the fetus develops and grows until birth. The uterus may develop uterine fibroids, adenomyosis, endometriosis, cervicitis, cervical dysplasia, cervical intraepithelial neoplasia, cervical cancer, uterine prolapse, and endometrial cancer, due to various factors. For research on these uterus-related diseases, technology for culturing organoids derived from uterine tissue is urgently needed.

Against this background, the present inventors have found that it is possible to culture endometrial organoids using extracellular matrix derived from decellularized uterine tissue, thereby completing the present invention.

### DISCLOSURE

### Technical Problem

An object of the present invention is to provide a hydrogel composition for three-dimensional culture of endometrial organoids, comprising decellularized uterine tissue-derived extracellular matrix (UEM).

Another object of the present invention is to provide endometrial organoids cultured in the hydrogel composition for three-dimensional culture.

Still another object of the present invention is to provide a method for preparing a hydrogel composition for three-dimensional culture of endometrial organoids, comprising steps of: mixing uterine tissue with Triton X-100 and ammonium hydroxide; preparing decellularized uterine tissue-derived tissue extracellular matrix by freeze-drying and grinding the tissue; solubilizing the decellularized uterine tissue-derived extracellular matrix in a pepsin solution; and mixing the solubilized extracellular matrix with PBS buffer, distilled water, and NaOH, followed by gelation.

Yet another object of the present invention is to provide a method for producing endometrial organoids, comprising a step of culturing endometrial organoids in the hydrogel composition for three-dimensional culture of endometrial organoids.

Still yet another object of the present invention is to provide a method of preventing or treating a uterus-related disease by transplanting the endometrial organoids into a subject having the uterus-related disease.

### Technical Solution

One aspect of the present invention provides a hydrogel composition for three-dimensional culture of endometrial organoids, comprising decellularized uterine tissue-derived extracellular matrix (UEM).

Another aspect of the present invention provides endometrial organoids cultured in the hydrogel composition for three-dimensional culture.

Still aspect of the present invention provides a method for preparing a hydrogel composition for three-dimensional culture of endometrial organoids, comprising steps of: mixing uterine tissue with Triton X-100 and ammonium hydroxide; preparing decellularized uterine tissue-derived extracellular matrix by freeze-drying and grinding the tissue; solubilizing the decellularized uterine tissue-derived extracellular matrix in a pepsin solution; and mixing the solubilized extracellular matrix with PBS buffer, distilled water, and NaOH, followed by gelation.

Yet another aspect of the present invention provides a method for producing endometrial organoids, comprising a step of culturing endometrial organoids in the hydrogel composition for three-dimensional culture of endometrial organoids.

Still yet another aspect of the present invention provides a method of preventing or treating a uterus-related disease by transplanting the endometrial organoids into a subject having the uterus-related disease.

### Advantageous Effects

The hydrogel composition comprising decellularized uterine tissue-derived extracellular matrix according to the present invention may be used to produce endometrial organoids having a very high similarity to in vivo uterine tissue and organs through the characteristics, components, and physical properties of the decellularized uterine tissue-derived extracellular matrix. These endometrial organoids may be useful for in vivo transplantation and testing for drugs for various uterus-related diseases.

### BRIEF DESCRIPTION OF DRAWINGS

FIG. 1 relates to the preparation of decellularized uterine tissue-derived uterus extracellular matrix.

FIGS. 2 to 5 relate to the components of the prepared decellularized uterine tissue-derived extracellular matrix (UEM).

FIGS. 6 to 9 relate to proteomic analysis of the prepared decellularized uterine tissue-derived extracellular matrix (UEM).

FIG. 10 relates to the solubility of decellularized uterine tissue-derived extracellular matrix (UEM) and the degree of hydrogel formation, depending on solubilization temperature.

FIGS. 11 and 12 relate to the results of analyzing the physical properties of a decellularized uterine tissue-derived extracellular matrix (UEM) hydrogel.

FIGS. 13 and 14 relate to the concentration of a decellularized uterine tissue-derived extracellular matrix (UEM) hydrogel, optimized for culture of endometrial organoids.

FIGS. 15 to 18 relate to the expression of endometrium-specific proteins in endometrial organoids cultured in the UEM hydrogel.

FIG. 19 relates to the analysis of expression of stem cell differentiation markers in endometrial organoids cultured in the UEM hydrogel.

FIGS. 20 and 21 relate to the solubilization concentration of decellularized uterine tissue-derived extracellular matrix (UEM), optimized for culture of endometrial organoids.

FIGS. 22 and 23 relate to the results of functional analysis of endometrial organoids cultured in a decellularized uterine tissue-derived extracellular matrix (UEM) hydrogel.

FIGS. 24 and 25 relate to changes in the morphology of endometrial organoids, cultured in a decellularized uterine tissue-derived extracellular matrix (UEM) hydrogel, depending on the concentration of WNT3a conditioned medium (CM).

FIGS. 26 and 27 relate to the results of analysis of differences between batches of a decellularized uterine tissue-derived extracellular matrix (UEM) hydrogel.

FIG. 28 relates to the results of investigating the similarity of organoid culture performance between batches of a decellularized uterine tissue-derived extracellular matrix (UEM) hydrogel.

FIGS. 29 to 32 relate to tissue-specific effects of decellularized uterine tissue-derived scaffolds for endometrial organoid culture.

FIG. 33 relates to passaging of endometrial organoids on decellularized uterine tissue-derived hydrogel scaffolds.

FIG. 34 relates to the results of establishing an animal model for in vivo transplantation of endometrial organoids.

FIG. 35 relates to the results of evaluating the in vivo transplantation and regeneration effect of endometrial organoids using a decellularized uterine tissue-derived hydrogel scaffold.

FIG. 36 relates to the results of verifying the applicability of a decellularized uterine tissue-derived extracellular matrix composition as a coating material.

FIGS. 37 to 42 relate to the fabrication of an endometrial organoid chip using a decellularized uterine tissue-derived hydrogel scaffold.

### BEST MODE

Hereinafter, the present invention will be described with reference to the accompanying drawings. The present invention may, however, be embodied in many different forms, and thus is not to be construed as being limited to the embodiments described herein. When any part is referred to as “comprising” or “containing” any component, it does not exclude other components, but may further comprise or contain other components, unless otherwise specified.

Unless otherwise defined, the practice of the present invention involves conventional techniques commonly used in molecular biology, microbiology, protein purification, protein engineering, protein and DNA sequencing, and recombinant DNA fields, which are within the skill of the art. Such techniques are known to those of skill in the art and are described in numerous texts and reference works.

Unless otherwise defined, all technical and scientific terms used have the same meanings as commonly understood by those skilled in the art.

Various scientific dictionaries that include the terms included herein are well known and available in the art. Although any method and material similar or equivalent to those described herein find use in the practice or testing of the present invention, some methods and materials are described. The present invention is not limited to particular methodology, protocols, and reagents, as these may vary depending upon the context to be used by those skilled in the art. Hereinafter, the present invention will be described in more detail.

The present invention provides a hydrogel composition for three-dimensional culture of endometrial organoids, comprising decellularized uterine tissue-derived extracellular matrix (UEM).

In another aspect of the present invention, the present invention provides endometrial organoids cultured in the hydrogel composition for three-dimensional culture.

In still another aspect of the present invention, the present invention provides a scaffold support for in vivo organoid transplantation, comprising the hydrogel composition for three-dimensional culture.

In yet another aspect of the present invention, the present invention provides a composition for coating the surface of a culture vessel, comprising the hydrogel composition for three-dimensional culture.

In still yet another aspect of the present invention, the present invention provides an endometrial organoid chip or a method of fabricating the same, comprising the hydrogel composition for three-dimensional culture.

The “extracellular matrix” refers to a natural scaffold for cell growth prepared through decellularization of the tissue found in mammals and multicellular organisms. The extracellular matrix may be further processed through dialysis or crosslinking.

The extracellular matrix may be a mixture of structural and non-structural biomolecules, including, but not limited to, collagens, elastins, laminins, glycosaminoglycans, proteoglycans, antimicrobials, chemoattractants, cytokines, and growth factors.

Decellularized uterine tissue contains actual tissue-specific extracellular matrix components, and thus may provide the physical, mechanical, and biochemical environment of the tissue in question, and is highly efficient in enhancing differentiation into uterine tissue cells and tissue-specific functionality.

The term “organoid” refers to an ultraminiature body organ prepared in the form of an artificial organ by 3D-culturing cells derived from tissues or pluripotent stem cells.

The organoid is a three-dimensional tissue analog that contains organ-specific cells which originate from stem cells and self-organize (or self-pattern) in a manner similar to the situation in vivo. The organoid can be developed into a specific tissue by patterning limited factors (for example, growth factors).

The organoid can have the intrinsic physiological properties of the cells and also can have an anatomical structure that mimics the original state of a cell mixture (containing limited cell types but also the remaining stem cells and the neighboring physiological niche). A three-dimensional culture method allows the organoid to be better arranged in terms of cell to cell functions, and to have an organ-like form with functionality and a tissue-specific function.

The “hydrogel” is a material in which a liquid that contains water as a dispersion medium is hardened through the sol-gel phase transition to lose fluidity and to form a porous structure. The hydrogel can be formed by causing a hydrophilic polymer that has a three-dimensional network structure and a microcrystalline structure to contain water and to be expanded.

In the present invention, the decellularized uterine tissue-derived extracellular matrix may contain glycosaminoglycan, collagen, fibronectin and/or laminin. The decellularized uterine tissue-derived extracellular matrix of the present invention is characterized in that all of these extracellular matrix proteins are well preserved.

In the present invention, the decellularized uterine tissue-derived extracellular matrix may contain collagen type VI COL6A3, COL6A1, and/or COL6A2 as collagen, and fibrinogen FGA, FGB, and/or FGG as glycoprotein. As such, the decellularized uterine tissue-derived extracellular matrix of the present invention is characterized by the presence of various types of extracellular matrix and growth factor proteins.

In the present invention, the decellularized uterine tissue-derived extracellular matrix may have a higher elastic modulus (G′) than the viscous modulus (G″). Specifically, when the concentration of the decellularized uterine tissue-derived extracellular matrix is 1 to 8 mg/mL, the extracellular matrix may have an elastic modulus of 10¹ to 10² Pa and a viscous modulus of 10⁰ to 10¹ Pa. Thereby, the decellularized uterine tissue-derived extracellular matrix of the present invention is characterized by having appropriate elastic modulus and viscous modulus, which enables the formation of a stable polymer network inside the hydrogel.

In the present invention, the concentration of the decellularized uterine tissue-derived extracellular matrix in the hydrogel composition for three-dimensional culture of endometrial organoids may be 1 to 8 mg/mL or 3 to 7 mg/mL. This concentration of extracellular matrix corresponds to a range that can stably form endometrial organoids, similar to the case of commercially available Matrigel.

In the present invention, endometrial organoids cultured in the hydrogel composition for three-dimensional culture may express or overexpress ERα, E-cadherin, and/or cytokeratin (pan-cytokeratin; PanCK). More specifically, endometrial organoids cultured in the hydrogel composition for three-dimensional culture according to the present invention may exhibit expression levels of ERα, E-cadherin, and/or PanCK, which are 90 to 99% similar to either endometrial organoids cultured in Matrigel, or endometrial organoids cultured on hydrogel scaffolds derived from organs other than the uterus, such as esophagus, heart, intestine, liver, spinal cord, pancreas, bladder, or salivary glands. These proteins are endometrium-specific proteins, indicating that the hydrogel composition for three-dimensional culture according to the present invention is meaningful as a replacement for existing Matrigel for culturing endometrial organoids.

In the present invention, endometrial organoids cultured in the hydrogel composition for three-dimensional culture may express or overexpress Esr1, Lgr5, Foxa2, and/or Muc1 gene. More specifically, endometrial organoids cultured in the hydrogel composition for three-dimensional culture according to the present invention may overexpress Esr1, Lgr5, Foxa2, and/or Muc1 gene compared to either endometrial organoids cultured in Matrigel, or endometrial organoids cultured on hydrogel scaffolds derived from organs other than the uterus, such as esophagus, heart, intestine, liver, spinal cord, pancreas, bladder, or salivary glands. Through this expression or overexpression of stem cell differentiation markers, the composition of the present invention may be provided as a hydrogel composition for inducing endometrial organoid development and differentiation, which is more suitable than Matrigel.

In the present invention, when the endometrial organoids cultured in the hydrogel composition for three-dimensional culture are treated with estradiol and/or progesterone as sex hormones, mucin secretion from the endometrial organoids may be increased, and more specifically, mucin secretion from the endometrial organoids may be increased compared to that from endometrial organoids cultured in Matrigel.

In addition, in the present invention, when endometrial organoids cultured in the hydrogel composition for three-dimensional culture are treated with estradiol, the expression level of a cell proliferation marker (Ki67) therein may be increased. More specifically, the cell proliferation marker may be overexpressed in endometrial organoids treated with estradiol alone compared to endometrial organoids treated with estradiol and progesterone. This indicates that endometrial organoids cultured in the hydrogel composition for three-dimensional culture according to the present invention have higher potential than those cultured in Matrigel.

In another aspect, the present invention provides a method for preparing a hydrogel composition for three-dimensional culture of endometrial organoids, comprising steps of: mixing uterine tissue with Triton X-100 and ammonium hydroxide; preparing decellularized uterine tissue-derived tissue extracellular matrix by freeze-drying and grinding the tissue; solubilizing the decellularized uterine tissue-derived extracellular matrix in a pepsin solution; and mixing the solubilized matrix solution with PBS buffer, distilled water, and NaOH, followed by gelation.

In still another aspect of the present invention, the present invention provides a method for producing endometrial organoids, comprising a step of culturing endometrial organoids in the hydrogel composition for three-dimensional culture of endometrial organoids.

In the present invention, the step of solubilizing the decellularized uterine tissue-derived extracellular matrix in the pepsin solution may be performed at a temperature of 15 to 30° C. This temperature condition is related to the solubility of the decellularized uterine tissue-derived extracellular matrix (UEM) and the degree of hydrogel formation. If the above temperature condition is not satisfied, a problem may arise in that the uterine tissue-derived extracellular matrix is not solubilized normally or a hydrogel does not form well.

In the present invention, in the step of solubilizing the decellularized uterine tissue-derived extracellular matrix in the pepsin solution, the concentration of the pepsin solution may be 3 to 10 mg/mL, or 4 to 8 mg/mL. Specifically, when the concentration of UEM is 20 mg/mL, the concentration of the pepsin solution may be 3 to 10 mg/mL, or 4 to 8 mg/mL. This concentration of the pepsin solution is for efficient and stable culture of endometrial organoids. If the concentration of the pepsin solution is out of the above range, a problem may arise in that the gel shrinks during organoid culture.

In the present invention, the method for producing endometrial organoids may further comprise a step of passaging the endometrial organoids in WNT3a conditioned medium (CM), wherein the concentration of the WNT3a conditioned medium may be 5 to 30, 10 to 25, or 10 or 25% (volume/volume). When the concentration of the CM is 5 to 15 or 10%, the organoids may proliferate actively and develop into a dense form during passaging, and when the concentration of the CM is 20 to 30% or 25%, the organoid may develop into a cystic form due to induction of cell differentiation.

In the present invention, the method for producing endometrial organoids may further comprise a step of passaging the hydrogel composition for three-dimensional culture of endometrial organoids.

Another aspect of the present invention provides a method of transplanting the endometrial organoid into a subject with endometrial damage or uterus-related disease.

Still another aspect of the present invention provides a method of ameliorating or treating endometrial damage by transplanting the endometrial organoid into a subject with endometrial damage.

Yet another aspect of the present invention provides a method of ameliorating or treating uterus-related disease by transplanting the endometrial organoid into a subject with uterus-related disease.

Still yet another aspect of the present invention provides the use of the endometrial organoid for the prevention or treatment of uterine-related disease.

A further aspect of the present invention provides the use of the endometrial organoid for manufacturing a medicament for preventing or treating uterine-related disease.

In the present invention, the uterus-related disease may be endometriosis, uterine fibroids, cervical cancer, adenomyosis, vaginitis, ovarian cyst, endometrial cancer, uterine cancer, cervical dysplasia, endometritis, uterine prolapse, cervicitis, functional uterine bleeding, or abnormal uterine bleeding.

In the present invention, the term “subject” refers to animals, including humans.

Endometrial organoids may be cultured and produced through the hydrogel composition for three-dimensional culture of endometrial organoids comprising decellularized uterine tissue-derived extracellular matrix according to the present invention. These endometrial organoids have high similarity to in vivo uterine tissue in various aspects, and thus may be used for the above-mentioned purposes.

### MODE FOR INVENTION

Hereinafter, one or more specific examples will be described in more detail by way of examples. However, these examples are intended to illustrate one or more embodiments, and the scope of the present invention is not limited to these examples.

### Example 1: Preparation of Decellularized Uterine Tissue-Derived Extracellular Matrix (UEM)

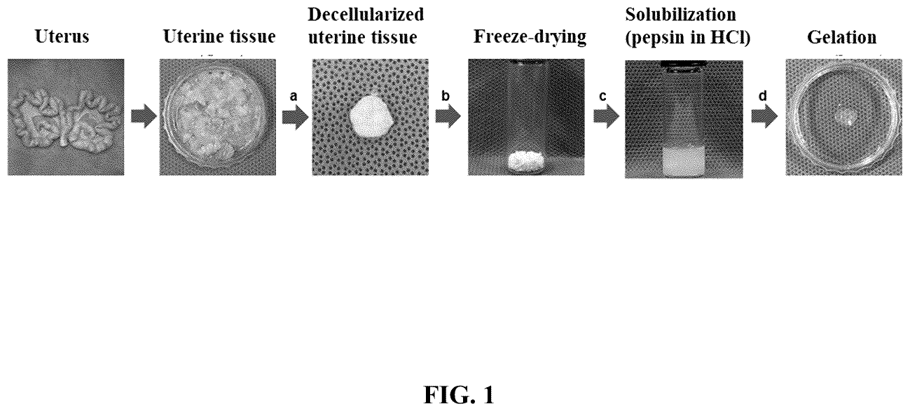

First, extracellular matrix derived from decellularized uterine tissue was prepared as shown in FIG. 1.

Specifically, porcine uterine tissue was cut into small pieces and then stirred in a mixture solution of 1% Triton X-100 and 0.1% ammonium hydroxide for 48 hours, thus removing all cellular components from the tissue (step a). After this decellularization process, the tissue was freeze-dried and ground, thereby preparing decellularized uterine tissue-derived extracellular matrix (UEM) (step b). 20 mg of the decellularized uterine tissue-derived extracellular matrix was solubilized in 4 mg/mL pepsin solution (a solution of 4 mg of porcine gastric mucosa-derived pepsin powder in 1 mL of 0.02 M HCl) for two days (step c).

UEM solution: 10×PBS buffer (pH 7.2):triple-distilled water:NaOH (0.5 M) were mixed together at a ratio of 25:10:63:2, stirred uniformly, and adjusted to a pH of 7.0 to 7.2, and then gelation of the mixture was induced at a temperature of 37° C. for 30 minutes, thereby preparing a scaffold composition in the form of a UEM hydrogel with a concentration of 5 mg/mL (step d).

### Example 2: Analysis of Decellularized Uterine Tissue-Derived Extracellular Matrix (UEM)

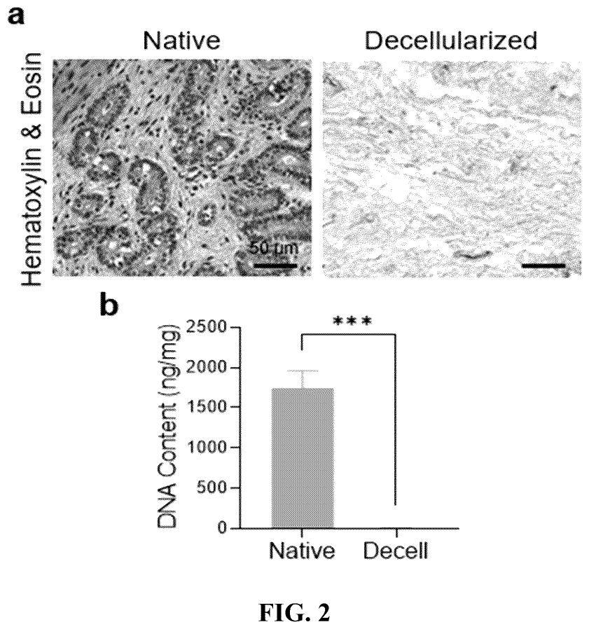

The characteristics of the prepared decellularized uterine tissue-derived extracellular matrix (UEM) were analyzed as shown in FIGS. 2 to 5.

(a and b in FIG. 2) It was confirmed through (a) H&E tissue staining and (b) DNA quantitative analysis before and after the decellularization process that most of the cellular components were removed by the decellularization process. It was confirmed that, compared to the uterine tissue before decellularization (native group), 99.5% of DNA was removed, and as the actual remaining amount, only a trace amount of DNA corresponding to a level of 8.95±1.28 ng per mg of freeze-dried UEM (decellularized group) remained.

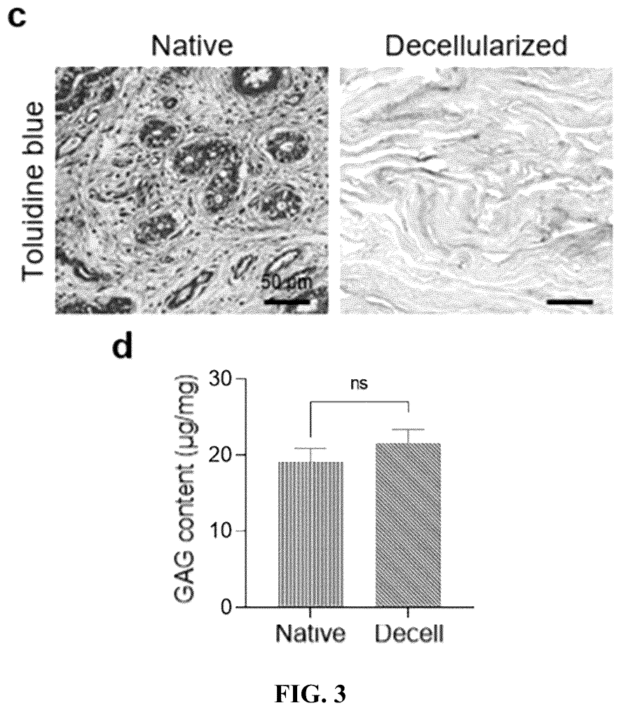

(c and d in FIG. 3) To analyze glycosaminoglycan (GAG), one of the representative extracellular matrix components, (c) toluidine blue tissue staining and (d) GAG quantitative analysis were performed. As a result, it was confirmed that GAG was well preserved in the decellularized uterine tissue.

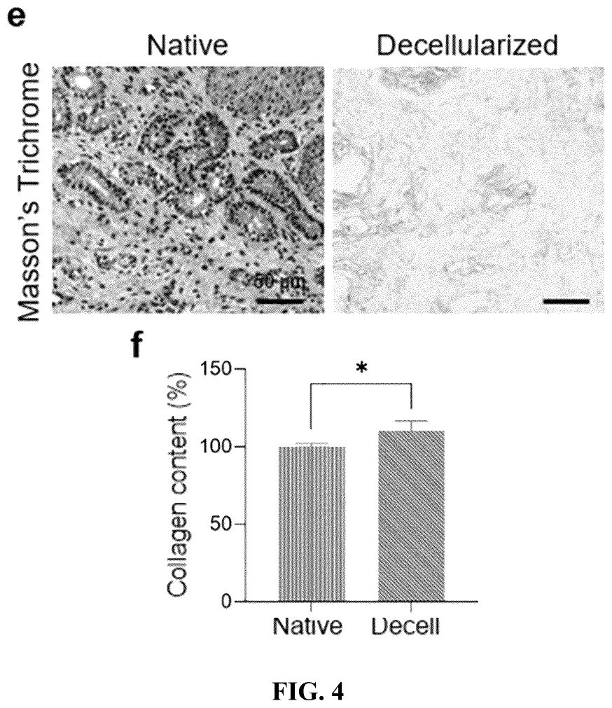

(e and f in FIG. 4) In addition, it was confirmed through (e) Masson's Trichrome tissue staining and (f) collagen quantitative analysis that collagen components were well preserved even after the decellularization process

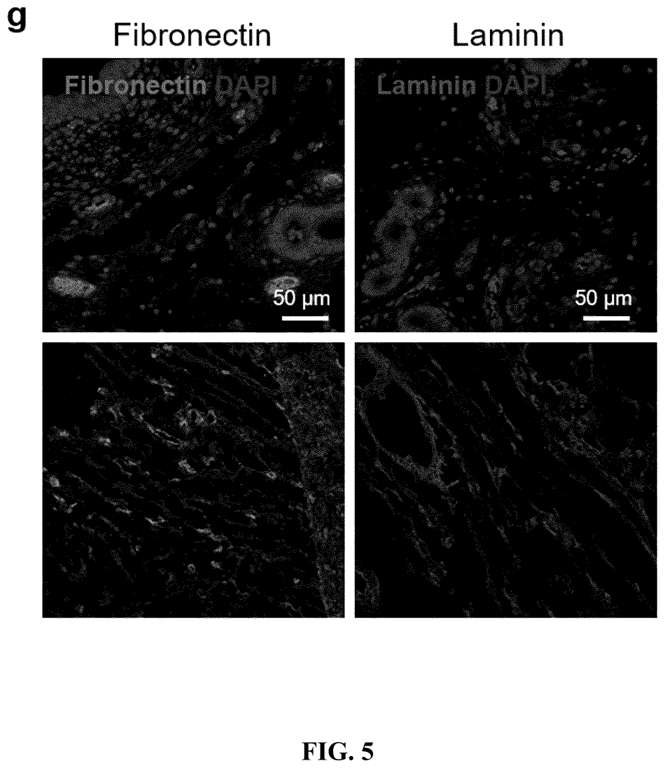

(g in FIG. 5) Through tissue immunostaining performed to confirm the presence of fibronectin and laminin, which are major extracellular matrix proteins, it was confirmed that the two extracellular matrix proteins were all well preserved even after the decellularization process. Also, it was confirmed through DAPI staining that all cell nuclei were removed.

### Example 3: Analysis of Components of Decellularized Uterine Tissue-Derived Extracellular Matrix (UEM)

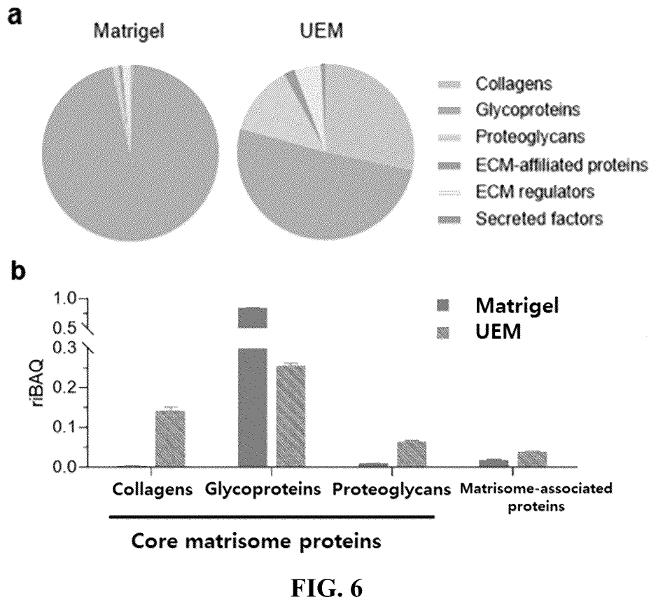

The components of the prepared decellularized uterine tissue-derived extracellular matrix (UEM) were analyzed as shown in FIGS. 6 to 9. Specifically, proteomics was analyzed by mass spectrometry to identify the components of the decellularized uterine tissue-derived extracellular matrix composition (UEM).

(a and b in FIG. 6) It could be seen that various types of extracellular matrix proteins (collagens, glycoproteins, proteoglycans, etc.) and growth factor proteins existed in UEM compared to Matrigel. On the other hand, it can be seen that Matrigel is mainly composed of glycoproteins.

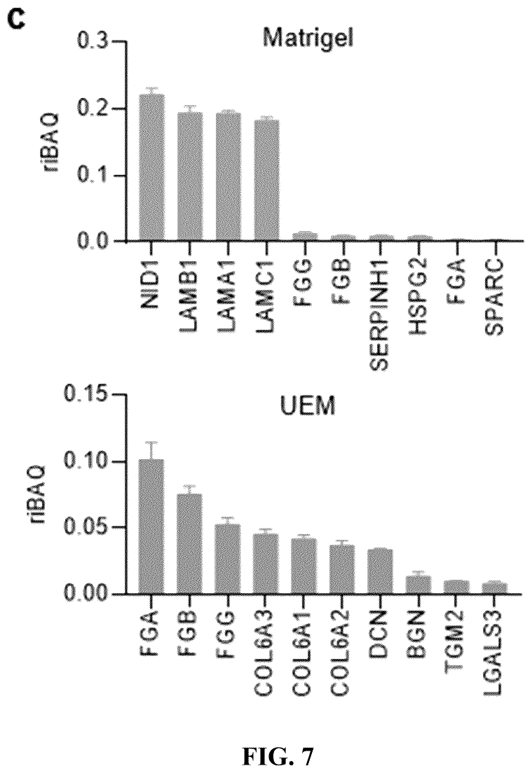

(c in FIG. 7) More specifically, it can be seen that the proteins expressed at the highest levels in UEM were various extracellular matrix components, including collagens [collagen type VI (COL6A3, COL6A1, and COL6A2)], glycoproteins [fibrinogens (FGA, FGB, and FGG)], and proteoglycans [decorin (DCN)], which were uniformly present, but in the case of Matrigel, glycoproteins [nidogen-1 (NID1), and laminin-111 (LAMB1, LAMA1, and LAMC1)] accounted for most of the components (total: 0.8 riBAQ).

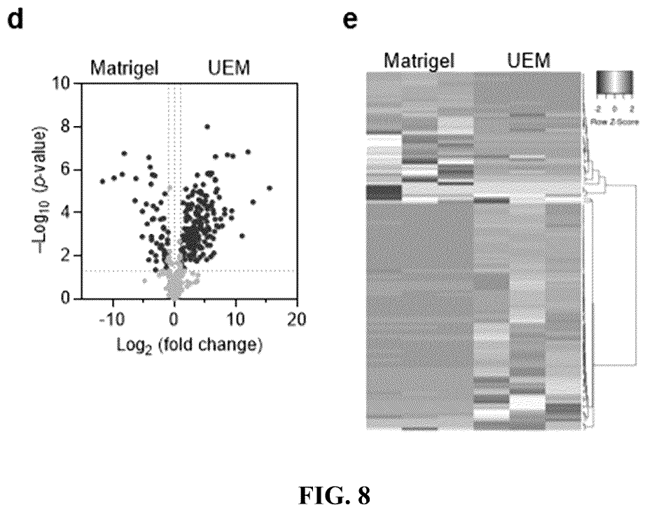

(d and e in FIG. 8) As a result of analyzing proteins whose expression levels are significantly different between Matrigel and UEM by (d) volcano plot and (e) heatmap, it could be seen that there were a number of proteins whose expression distributions are different between the two scaffolds.

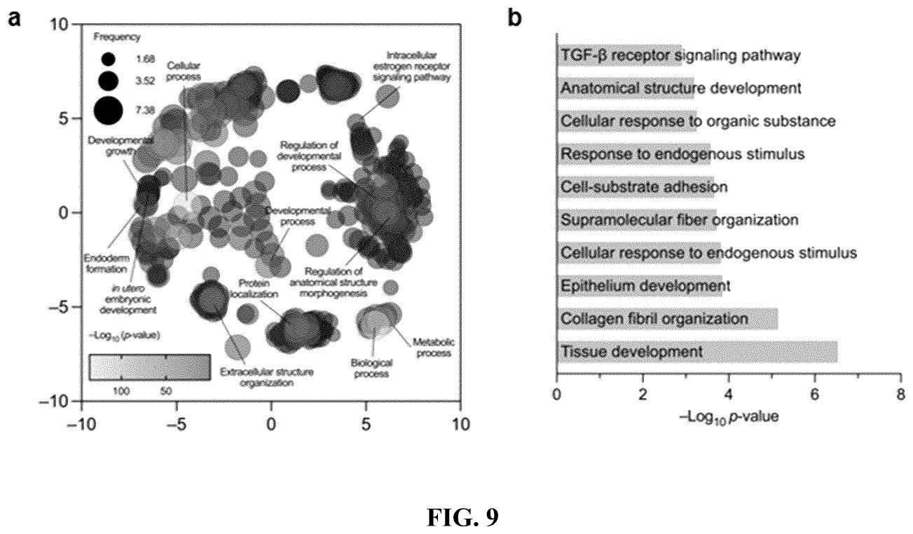

(a in FIG. 9) As a result of performing gene ontology analysis of all proteins present in UEM, it was confirmed that there were a number of proteins that mainly play roles related to structure formation, development, and morphogenesis. In particular, it can be seen that there are proteins related to uterus-specific development, maturation, and function, such as “intracellular estrogen signaling pathway”, and “in utero embryonic development”.

(b in FIG. 9) As a result of gene ontology analysis of proteins known to be significantly more expressed in the uterus than in other tissues, among the proteins present in UEM, it was confirmed that these proteins are mainly related to tissue development and morphogenesis.

### Example 4: Evaluation of Solubility and Hydrogel Formation of Decellularized Uterine Tissue-Derived Extracellular Matrix (UEM) Depending on Solubilization Temperature (Optimization of Step of Preparing Scaffold Solution)

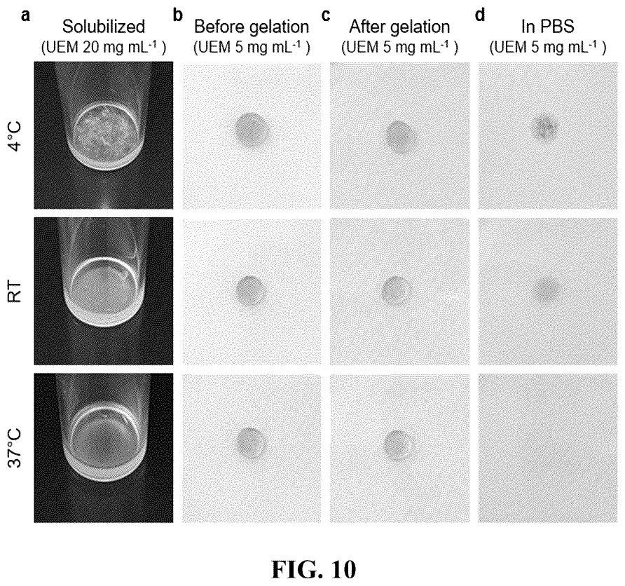

As shown in FIG. 10, the solubility and degree of hydrogel formation of the decellularized uterine tissue-derived extracellular matrix (UEM) were evaluated depending on solubilization temperature.

(a in FIG. 10) The dried decellularized uterine tissue-derived scaffold (20 mg/mL of UEM) was solubilized by treatment with pepsin (4 mg/mL) for two days under three temperature conditions. It was confirmed that, at 4° C., only a portion of the scaffold was solubilized, and at room temperature (RT) and 37° C., the scaffold was well solubilized (leftmost images).

(b in FIG. 10) The solubilized UEM (20 mg/mL) was mixed with triple-distilled water, 10×PBS (pH 7.2), and NaOH, thereby preparing a pre-gel UEM solution (pH 7.0 to 7.2) with a concentration of 5 mg/mL.

(c in FIG. 10) After inducing gelation at 37° C. for 30 minutes, observation was performed to examine whether a hydrogel was well formed. As shown in d in FIG. 10, when placed in a PBS buffer, the UEM sample solubilized at 4° C. formed a hydrogel but showed undissolved solid substances (top rightmost image), the UEM sample solubilized at room temperature formed a hydrogel well (rightmost middle image), and the UEM sample solubilized at 37° C. did not form a hydrogel (bottom rightmost image).

Therefore, it could be seen that the condition in which the decellularized uterine tissue-derived extracellular matrix (UEM) was solubilized by pepsin at room temperature was most suitable for forming the UEM hydrogel.

### Example 5: Analysis of Physical Properties of Decellularized Uterine Tissue-Derived Extracellular Matrix (UEM)

A hydrogel was produced by inducing crosslinking of the UEM solubilized by pepsin treatment at room temperature, and the mechanical properties thereof were analyzed.

Specifically, the physical properties of decellularized uterine tissue-derived extracellular matrix (UEM) hydrogel scaffolds at concentrations of 2, 3, 5 and 7 mg/mL were analyzed by measuring the elastic modulus (G′) and the viscous modulus (G″) at a frequency in the range of 0.1 to 10 Hz by means of a rotary rheometer.

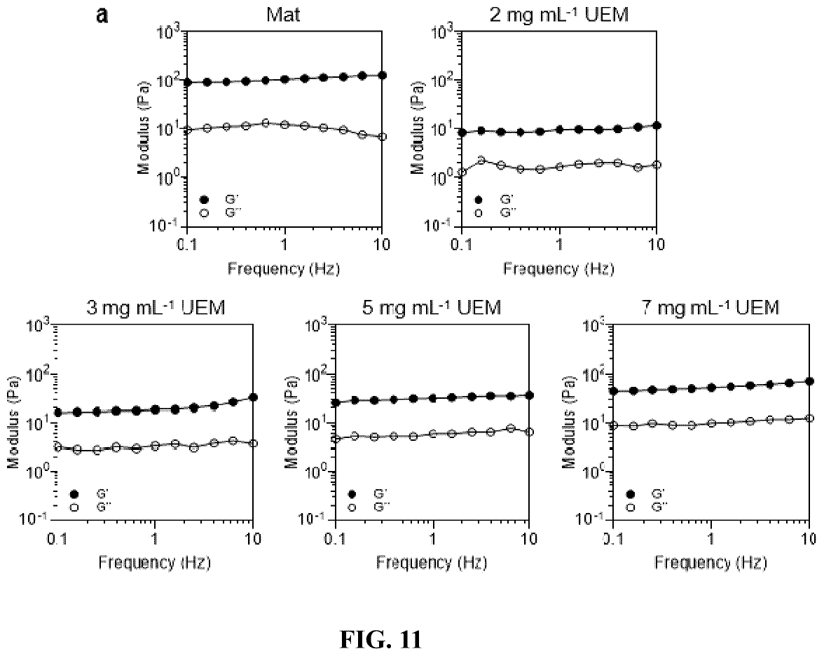

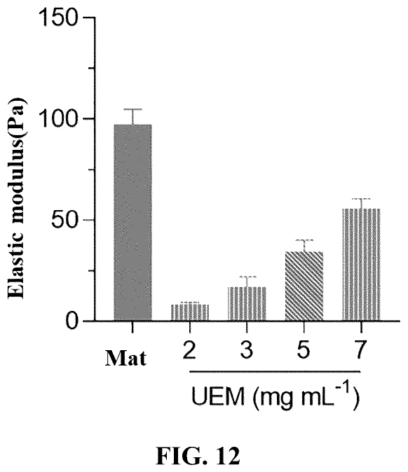

As a result, as shown in a in FIG. 11, it was confirmed that after 30 minutes of the crosslinking reaction, a stable polymer network was formed (G′>G″) in the UEM hydrogels prepared at all the concentrations. In addition, it was confirmed that the physical properties increased as the concentration of UEM increased (b in FIG. 12).

### Example 6: Determination of Concentration of Decellularized Uterine Tissue-derived Extracellular Matrix (UEM) Hydrogel Optimized for Culture of Endometrial Organoids

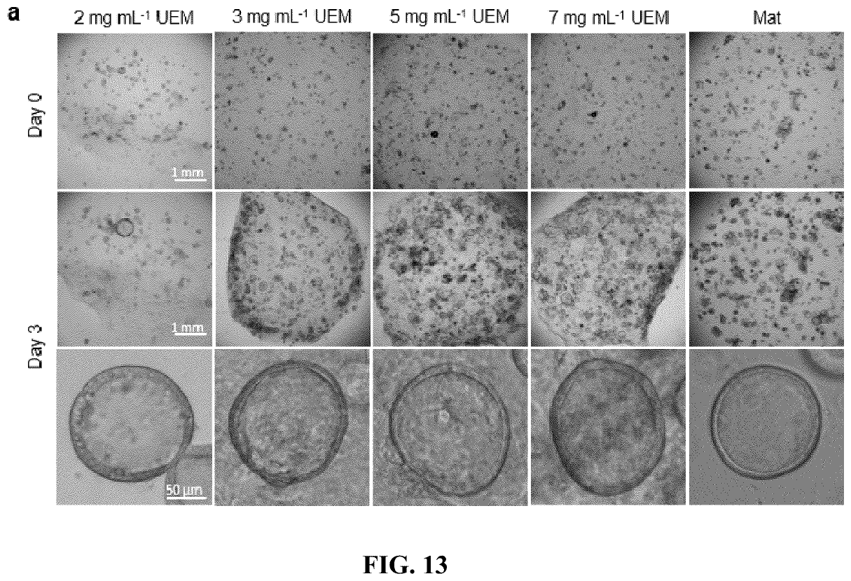

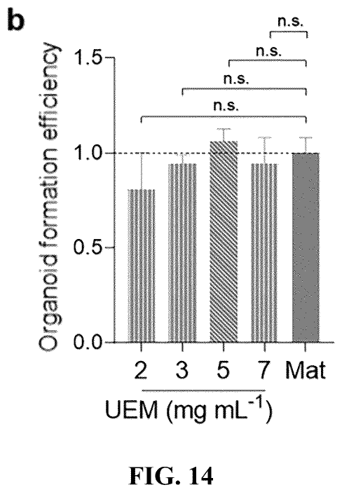

As shown in FIGS. 13 and 14, the present inventors sought to confirm the concentration of the decellularized uterine tissue-derived extracellular matrix (UEM) hydrogel, optimized for culture of endometrial organoids.

Specifically, fat and blood vessels were removed from mouse uterine tissue, and then cells were extracted by treatment with collagenase type V enzyme and cultured in an appropriate three-dimensional culture scaffold to form endometrial organoids. In order to select the optimal concentration of the UEM hydrogel scaffold, developed in the present invention, for culture of endometrial organoids, the endometrial organoids were cultured in each of UEM hydrogels at different concentrations (2, 3, 5, and 7 mg/mL) and the commercially available culture scaffold Matrigel (seeding density: 3.3×10⁶ cells/mL), and the morphology, formation efficiency, and gene expression of the formed endometrial organoids were compared.

As a result, it was confirmed that endometrial organoids were formed in all of the UEM hydrogels with four concentrations (2, 3, 5, and 7 mg/mL), and had a morphology similar to that of the organoids cultured in Matrigel (a in FIG. 13).

In addition, as a result of comparing the organoid formation efficiency of the UEM hydrogel at each concentration to that of Matrigel on day 3 of culture, it was observed that the organoid formation efficiency was similar between all conditions including Matrigel. Thereamong, the UEM hydrogel at 5 mg/mL was found to show the highest formation efficiency compared to the Matrigel and the UEM hydrogels at the other concentrations (b in FIG. 14).

### Example 7: Analysis of Expression of Endometrium-Specific Protein in Endometrial Organoids Cultured in UEM Hydrogel (Cell Immunostaining Assay)

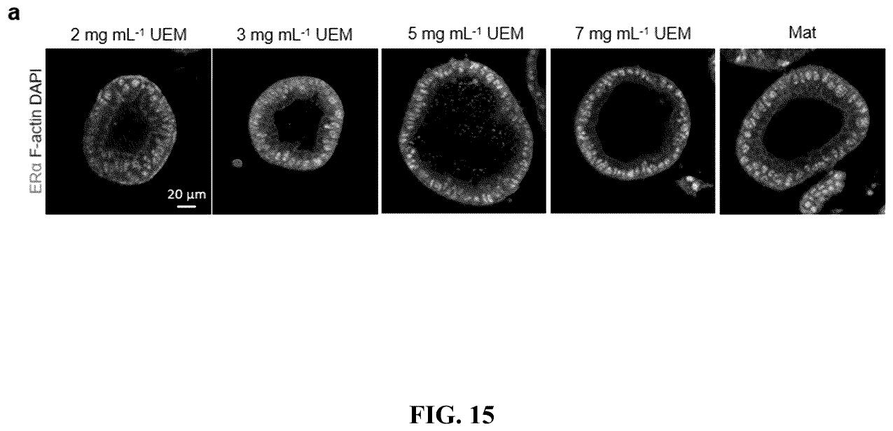

As shown in FIGS. 15 to 18, the expression of endometrium-specific proteins in the endometrial organoids cultured in the UEM hydrogel was analyzed.

As a result of performing immunofluorescence staining assay on day 4 of culture on the endometrial organoids cultured in each of Matrigel and the UEM hydrogels at various concentrations, it was confirmed that an estrogen receptor-alpha (ERα) marker, one of the types of nuclear receptors activated by estrogen, was well expressed in the organoids (FIG. 15a).

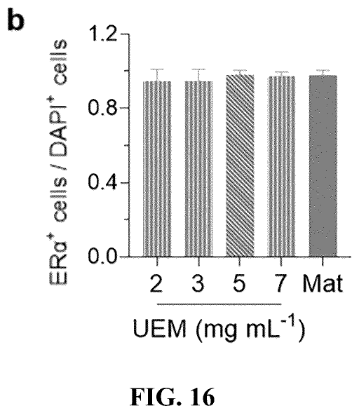

In addition, when the endometrial organoids cultured in each of Matrigel and the UEM hydrogels at various concentrations were analyzed on day 4 of culture, it was confirmed that estrogen receptor-alpha (ERα)-positive cells were distributed at similar levels (b in FIG. 16).

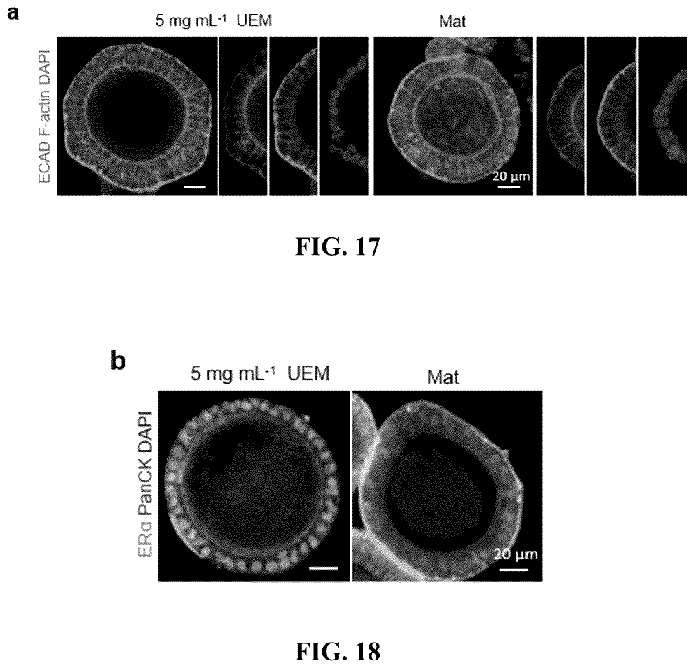

Next, the results of immunofluorescence staining assay performed on day 4 of culture in UEM indicated that the expression of the epithelial marker protein E-cadherin in the endometrial organoids cultured in the UEM hydrogel at a concentration of 5 mg/mL among the UEM hydrogels at various concentrations was similar to that in the organoids cultured in Matrigel (a in FIG. 17).

In addition, it was confirmed that the expression of estrogen receptor a (ERα) and the intermediate filament pan-cytokeratin (PanCK), which forms the cytoskeleton of epithelial cells, in the organoids cultured in the UEM hydrogel at a concentration of 5 mg/mL showed a pattern similar to that in the organoids cultured in Matrigel (b in FIG. 18).

Therefore, it can be seen that the UEM hydrogel at a concentration of 5 mg/mL has potential as a replacement for existing Matrigel for culturing endometrial organoids.

### Example 8: Analysis of Expression of Stem Cell Differentiation Markers in Endometrial Organoids Cultured in UEM Hydrogel (Quantitative PCR Analysis)

The mRNA expression level for a specific gene in the endometrial organoids cultured in each of the decellularized uterine tissue-derived UEM hydrogels at three concentrations (3, 5 and 7 mg/mL) and Matrigel was analyzed by quantitative PCR analysis on day 4 of culture.

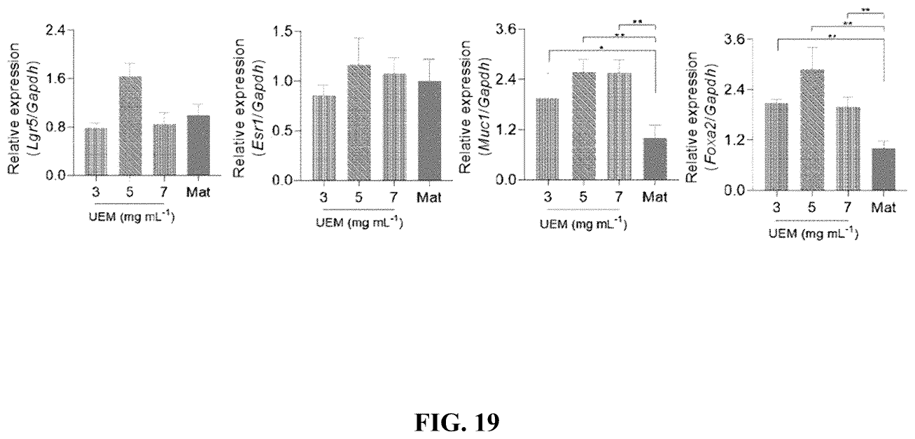

As a result, as shown in FIG. 19, it could be confirmed that the estrogen receptor alpha gene Esr1 and the stemness-related gene Lgr5 in the 5 mg/mL UEM endometrial organoid group were expressed at higher levels than those in the 3 mg/mL and 7 mg/mL UEM hydrogel groups and the Matrigel group.

In addition, when Foxa2, a gene related to the direct regulation of uterine gland development, and Mucin-1 (Muc1), a main component which is produced by epithelial cells and provides viscosity to cell membrane mucus, were compared between the endometrial organoids cultured in the UEM hydrogel and the organoids cultured in Matrigel, it was confirmed that the expression of the relevant markers increased in the UEM hydrogel groups at all the concentrations compared to the Matrigel group.

These results suggest that the UEM hydrogel has the ability to induce endometrial organoid development and differentiation, indicating that it can replace the commercially available Matrigel scaffold.

### Example 9: Determination of Solubilization Concentration of Decellularized Uterine Tissue-Derived Extracellular Matrix (UEM) Optimized for Culture of Endometrial Organoids

In order to determine the optimal solubilization condition of UEM for culture of endometrial organoids, UEM was solubilized for two days by treatment with various concentrations (2, 4 and 8 mg/mL) of pepsin. To prepare the solubilized UEM into a hydrogel, the UEM concentration was set to 5 mg/mL, which was previously determined as the optimized UEM concentration.

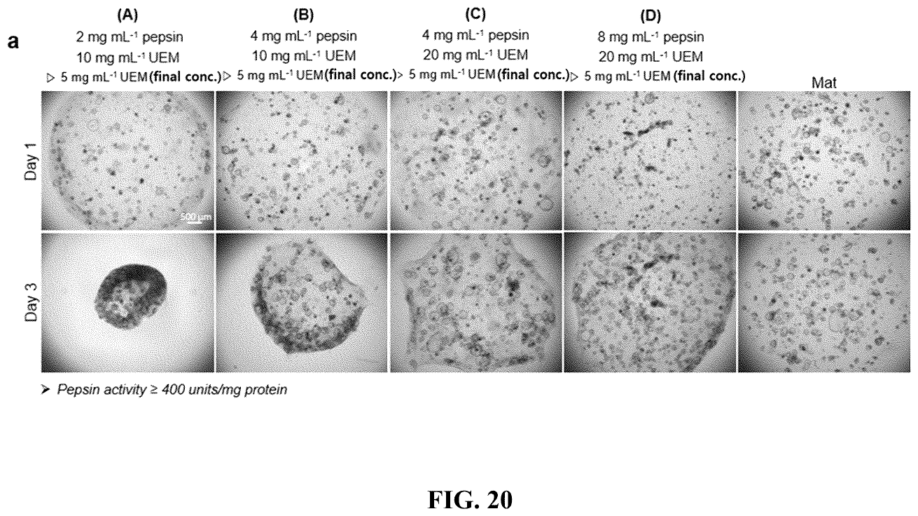

(a in FIG. 20) UEM was solubilized under four conditions (A: solubilization of 10 mg/mL UEM by 2 mg/mL pepsin, B: solubilization of 10 mg/mL UEM by 4 mg/mL pepsin, C: solubilization of 20 mg/mL UEM by 4 mg/mL pepsin, and D: solubilization of 20 mg/mL UEM by 8 mg/mL pepsin). In all the groups, the final concentration was adjusted to 5 mg/mL UEM, and formation of endometrial organoids was induced (seeding density: 3.3×10⁶ cells/mL). As a result, endometrial organoids were formed under all the conditions, but in conditions A and B, it was observed that the gel shrank when the organoids were cultured for 3 days or more.

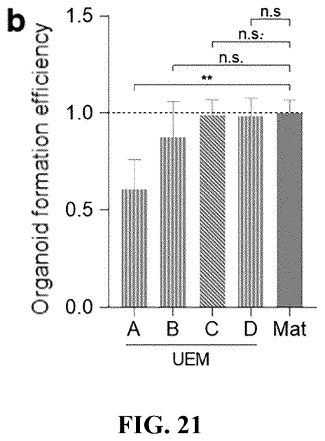

(b in FIG. 21) As a result of comparing the organoid formation efficiency between the UEM solubilization conditions on day 3 of culture, it was confirmed that endometrial organoids were formed in the UEM hydrogels under conditions C and D with the efficiency most similar to that of Matrigel. Therefore, the most appropriate protocols for solubilizing UEM were conditions C and D, and thereamong, condition C, in which the pepsin concentration is low, was finally selected.

### Example 10: Analysis of Functionality (Sex Hormone Responsiveness) of Endometrial Organoids Cultured in Decellularized Uterine Tissue-Derived Extracellular Matrix (UEM)

Sex hormone responsiveness was analyzed to evaluate the functionality of endometrial organoids cultured in the decellularized uterine tissue-derived UEM hydrogel having a concentration of 5 mg/mL. Specifically, endometrial organoids (seeding density: 3.3×10⁶ cells/mL) cultured for one day in Matrigel or the UEM hydrogel were treated with sex hormones.

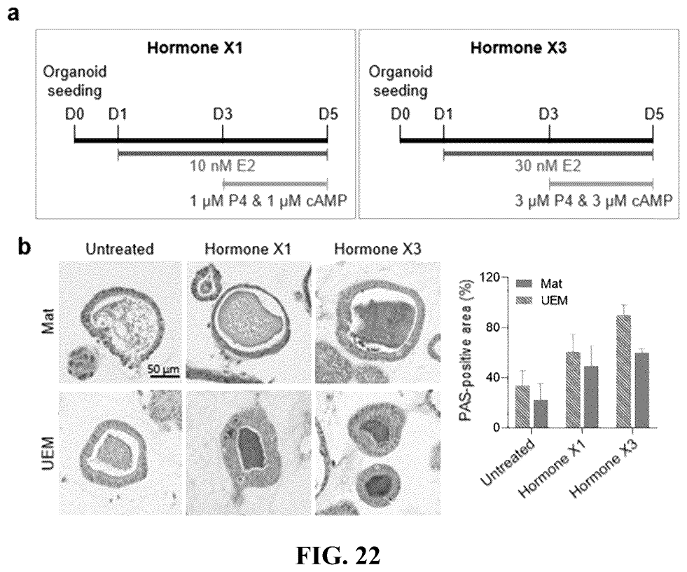

(a in FIG. 22) A hormone X1 group was treated with 10 nM estradiol (E2) for 2 days (D1-D3), and then further treated with 10 nM estradiol (E2), 1 μM progesterone (P4), and 1 μM cAMP for 2 days (D3-D5). A hormone X3 group was treated with 30 nM estradiol (E2) for 2 days (D1-D3), and then further treated with 30 nM estradiol (E2), 3 μM progesterone (P4), and 3 μM cAMP for 2 days (D3-D5).

(b in FIG. 22) Day 4 (D5) after sex hormone treatment, mucin secretion from the endometrial organoids by sex hormone treatment was determined by performing PAS staining and measuring the PAS-positive area in the organoid. The amount of mucin secretion significantly increased in proportion to the sex hormone treatment concentration in endometrial organoids cultured in each of Matrigel and the UEM hydrogel. In particular, it was confirmed that the UEM organoids secreted more mucin than the Matrigel organoids when treated with the same concentration of sex hormones.

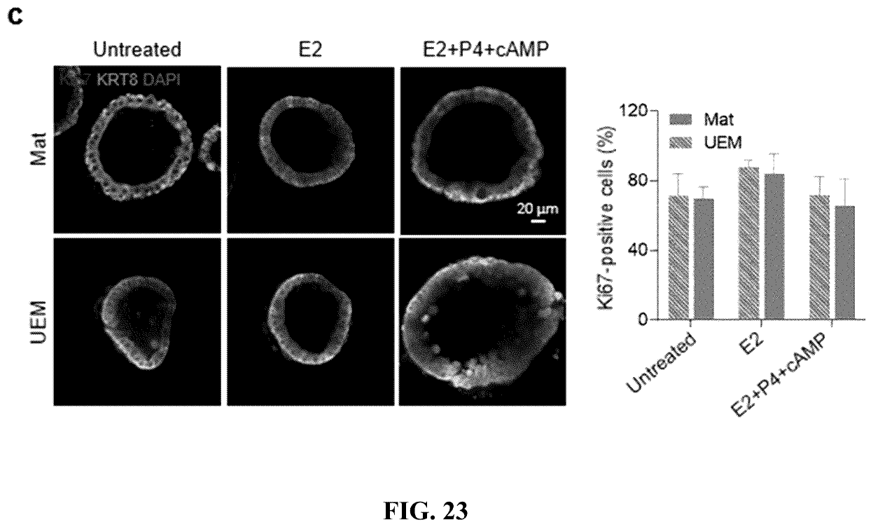

(c in FIG. 23) To examine the responsiveness of endometrial organoids depending on the sex hormone secretion cycle, immunofluorescence staining was performed on a group not treated with sex hormones (untreated), a group treated with 10 nM estradiol alone for 4 days (E2), and a group treated with 10 nM estradiol for 2 days and then further treated with 10 nM estradiol, 1 μM progesterone, and 1 μM CAMP for 2 days (E2+P4+cAMP). Treatment with estradiol alone induces cell proliferation in uterine tissue, and accordingly, the expression of the cell proliferation marker Ki67 was observed to be higher in the group treated with estradiol alone (E2) than in the other groups. Progesterone, which induces the secretory phase, induces cell differentiation in uterine tissue, and at this time, cAMP assists progesterone to help induce cell differentiation, and accordingly, the distribution of Ki67-positive cells decreased. On the other hand, Cytokeratin 8 (KRT8), an epithelial cell marker unrelated to hormone treatment, was expressed similarly in all of the three groups.

Thereby, it was verified that endometrial organoids cultured in the UEM hydrogel had functionality similar to or slightly higher than compared to those cultured in Matrigel.

### Example 11: Changes in Morphology of Endometrial Organoids, Cultured in Decellularized Uterine Tissue-Derived Extracellular Matrix (UEM) Hydrogel, Depending on Concentration of WNT3a Conditioned Medium (CM)

Endometrial organoids were passaged on days 4, 8 and 12 (P1, P2, and P3) in growth media containing WNT3a conditioned medium (CM) at 10% (volume/volume) and 25% (volume/volume), respectively, and changes in morphology of the endometrial organoids, cultured in 5 mg/mL of the decellularized uterine tissue-derived UEM hydrogel, depending on the concentration of WNT3a CM, were examined on days 7, 11, and 15 of culture.

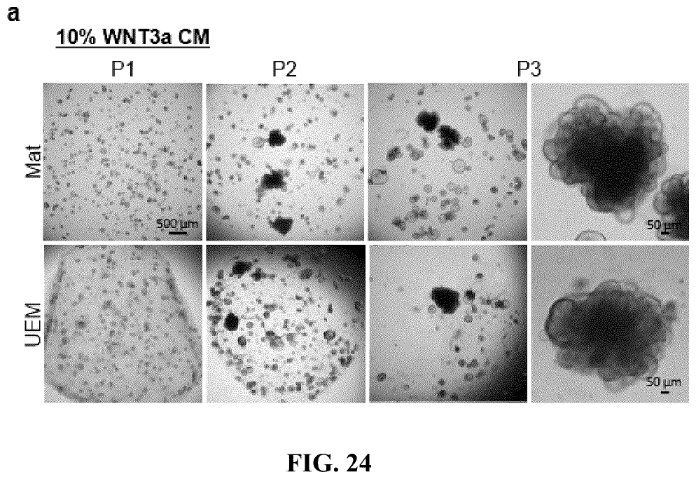

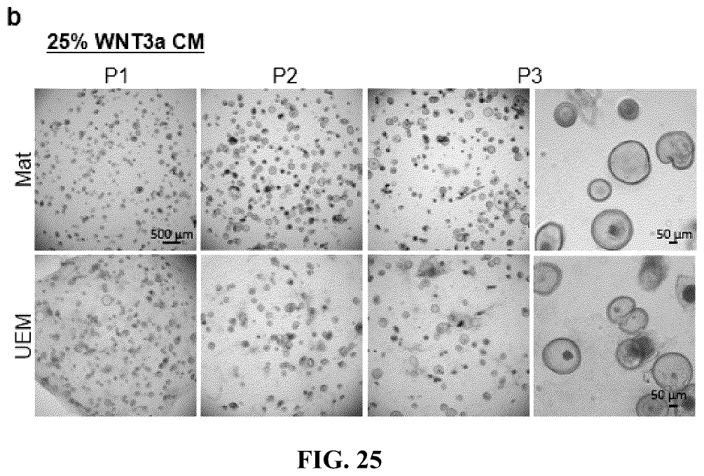

(a in FIG. 24 and b in FIG. 25) Endometrial organoids cultured under the 10% WNT3a CM condition developed into a dense form through passaging, and endometrial organoids cultured under the 25% WNT3a CM conditions developed into a cystic form. Endometrial organoids cultured in the UEM hydrogel developed in response to both WNT3a CM concentrations in a manner similar to those in Matrigel. Specifically, it was confirmed that, under the 10% WNT3a CM condition, cell proliferation occurred actively and the organoids developed into a dense form, and under the 25% WNT3a CM condition, cell differentiation was induced and the organoids developed into a cystic form.

### Example 12: Analysis of Whether Decellularized Uterine Tissue-Derived Extracellular Matrix (UEM) Hydrogel is Different Between Batches

Endometrial organoids were cultured in 5 mg/mL of each of UEM hydrogels (batches 1, 2 and 3) formed of UEM compositions prepared through a decellularization process from different porcine-derived uterine tissues, and similarity between the batches was checked.

Specifically, for culture of endometrial organoids, cells were isolated from mouse uterine tissue, cultured for 4 days at a cell density of 3.3×10⁶ cells/mL, and then passaged in the UEM hydrogel at a ratio of 1:3.

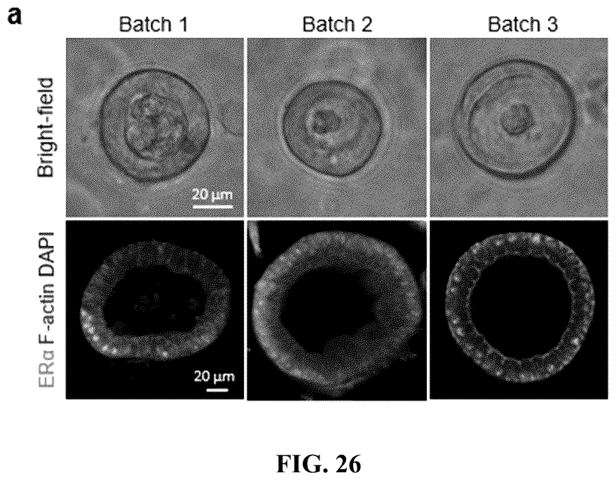

(a in FIG. 26) It was confirmed that all endometrial organoids cultured in three different UEM hydrogel batches for 4 days had similar morphologies. In addition, as a result of comparing the expression of estrogen receptor alpha (ERα) and F-actin, a major component of the cytoskeleton, through immunofluorescence staining, it was confirmed that the relevant proteins were expressed in all the three batches in similar patterns.

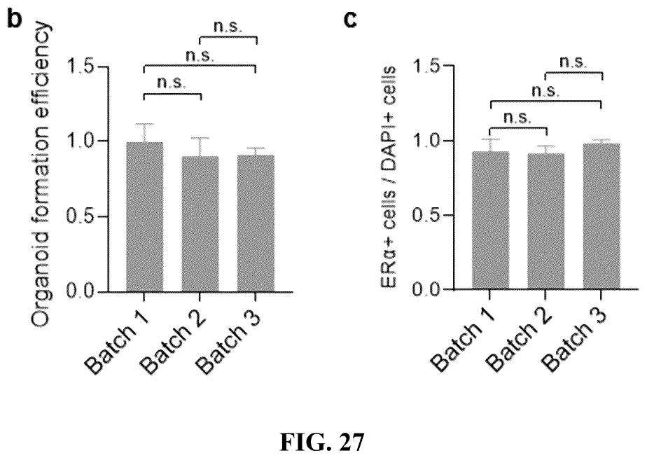

(b in FIG. 27) As a result of comparing the organoid formation efficiency on day 3 of culture in the UEM hydrogels, it was confirmed that the organoid formation efficiency was similar between the batches.

(c in FIG. 27) The number of ERα-positive cells was similarly distributed in the endometrial organoids cultured in the three different UEM hydrogel batches.

### Example 13: Examination of Similarity of Organoid Culture Performance Between Decellularized Uterine Tissue-Derived Extracellular Matrix (UEM) Hydrogel Batches

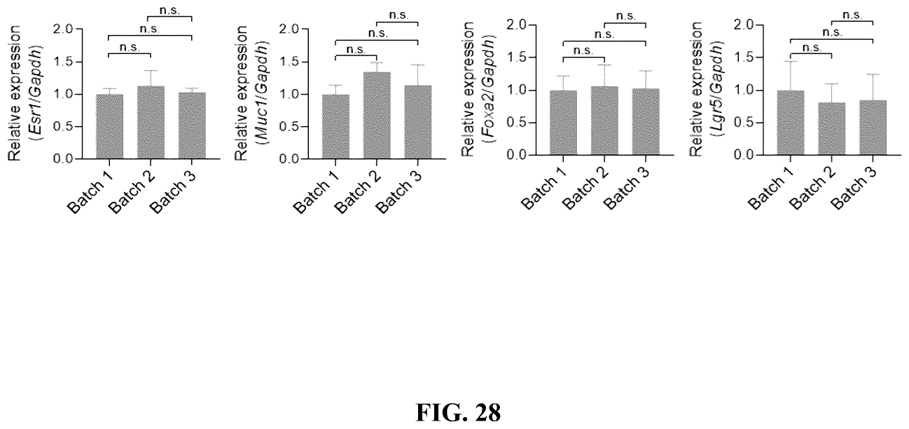

As shown in FIG. 28, as a result of performing quantitative PCR analysis on day 4 of culture on the endometrial organoids cultured in 5 mg/mL UEM hydrogel of each batch, it was confirmed that the expression levels of Lgr5, a gene related to stemness, and Foxa2, a gene that regulates uterine gland development, were similar between the batches. In addition, the expression levels of Esr1, an estrogen receptor alpha expression gene, and Mucin-1 (Muc1), which constitutes the epithelial cell mucosa, were not significantly different between the batches.

Therefore, it can be seen that endometrial organoid culture and development are possible in the same manner even when different UEM batches are used, and it can be inferred that it is possible to produce endometrial organoids at a uniform level through culture using a UEM-based hydrogel obtained through a decellularization process.

### Example 14: Evaluation of Tissue-Specific Effect of Decellularized Uterine Tissue-Derived Scaffold for Culture of Endometrial Organoids

To confirm that the UEM hydrogel scaffold provides a tissue-specific microenvironment for culturing endometrial organoids, endometrial organoids were also cultured in extracellular matrix hydrogels derived from decellularized tissues of different organs, and organoid formation and marker expression patterns were compared with those in organoids cultured in the UEM hydrogel scaffold. For culture of endometrial organoids, cells were isolated from mouse uterine tissue, cultured in Matrigel at a cell density of 3.3×10⁶ cells/mL for 4 days, and then passaged at a ratio of 1:3. At this time, in a decellularized salivary gland tissue-derived extracellular matrix hydrogel, endometrial organoids were cultured at a concentration of 7 mg/mL, and in other decellularized tissue-derived extracellular matrix hydrogels, including UEM, endometrial organoids were cultured at a concentration of 5 mg/mL.

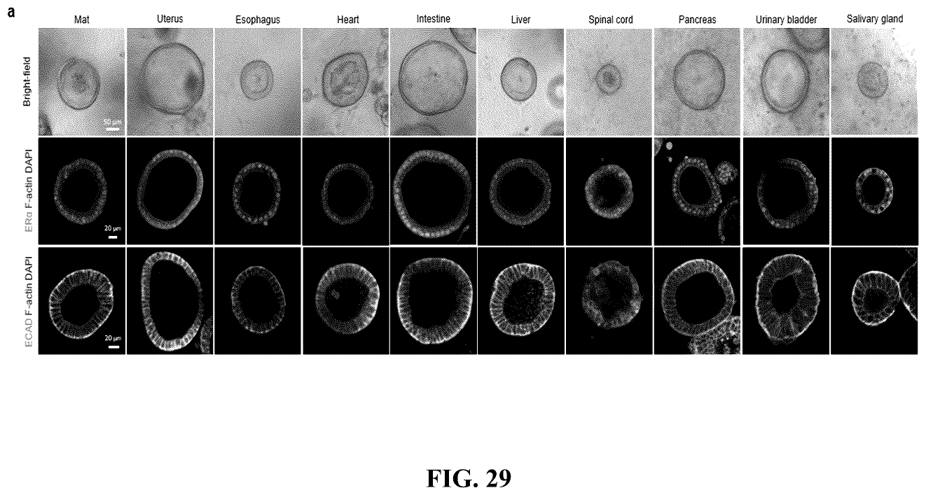

(a in FIG. 29) It was confirmed that endometrial organoids were formed in all of hydrogel scaffolds derived from decellularized uterus, esophagus, heart, intestines, liver, spinal cord, pancreas, bladder, and salivary glands, although there were differences in appearance between the hydrogel scaffolds. As a result of comparing the expression levels of estrogen receptor alpha, F-actin, and E-cadherin through immunofluorescence staining on day 4 of culture in the decellularized tissue-derived extracellular matrix hydrogels, it was confirmed that the expression of E-cadherin decreased the most in the spinal cord-derived hydrogel scaffold among various tissue-derived scaffolds, and the expression of estrogen receptor alpha was observed to be the lowest in the salivary gland tissue-derived scaffold.

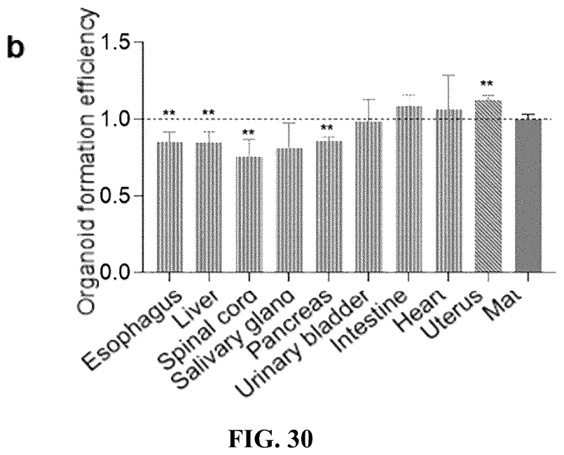

(b in FIG. 30) As a result of comparing the organoid formation efficiency between the decellularized tissue-derived hydrogel scaffolds on day 3 of culture, it was shown that the endometrial organoid formation efficiency was lower in almost all tissue-derived hydrogel scaffolds than in the UEM hydrogel.

This result shows that the UEM scaffold is most suitable for culture of endometrial organoids and the UEM scaffold has a tissue-specific effect.

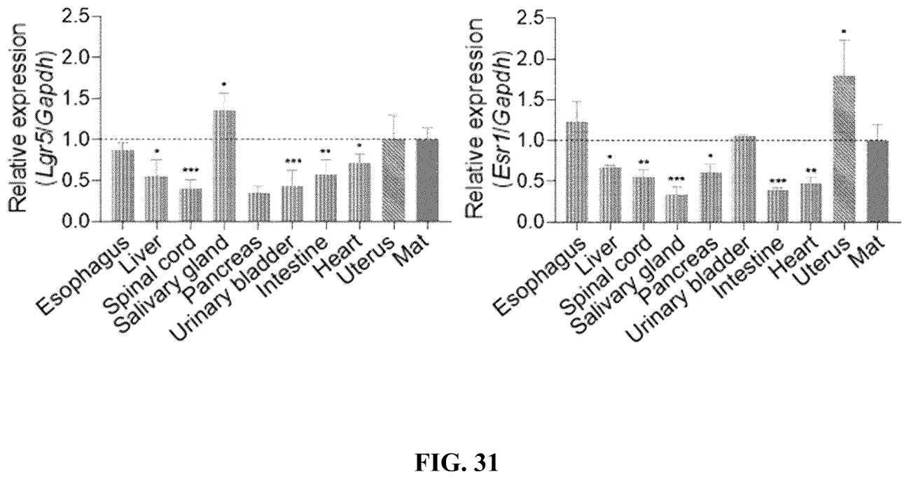

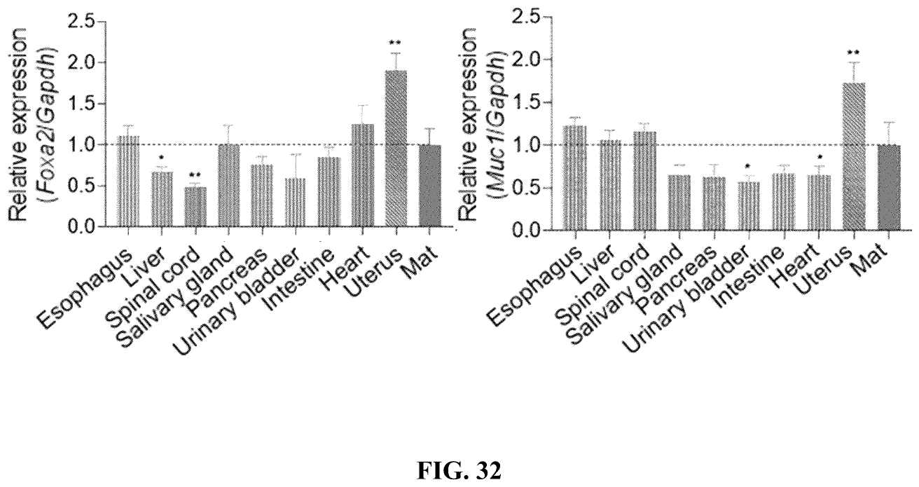

Next, as shown in FIGS. 31 and 32, in order to analyze the tissue-specific effect of the UEM hydrogel for culture of endometrial organoids, quantitative PCR analysis for endometrial organoids cultured in extracellular matrix hydrogels derived from decellularized tissues of various organs was performed on day 4 of culture, and the expression of each marker was comparatively analyzed. In the decellularized salivary gland tissue-derived extracellular matrix hydrogel, endometrial organoids were cultured at a concentration of 7 mg/mL, and in other decellularized tissue-derived extracellular matrix hydrogels, including UEM, endometrial organoids were cultured at a concentration of 5 mg/mL.

It was shown that the expression of Lgr5, a gene related to stemness, was lower when cultured in all the tissue-derived scaffolds (except salivary gland tissue-derived scaffold) than when cultured in UEM. In addition, it was shown that the expression levels of Esr1, an estrogen receptor alpha expression gene, Foxa2, a uterine gland development regulatory gene, and Muc1, an epithelial mucosal mucin expression gene, were lower when cultured in all the tissue-derived scaffolds than when cultured in the UEM scaffold. In addition, organoids cultured in the UEM hydrogel showed similar or higher expression of each marker gene compared to those cultured in Matrigel or the other tissue-derived scaffolds.

Therefore, it was confirmed that the UEM hydrogel scaffold can provide the most suitable microenvironment for the formation and development of endometrial organoids.

### Example 15: Passaging of Endometrial Organoids in Decellularized Uterine Tissue-Derived Hydrogel Scaffold

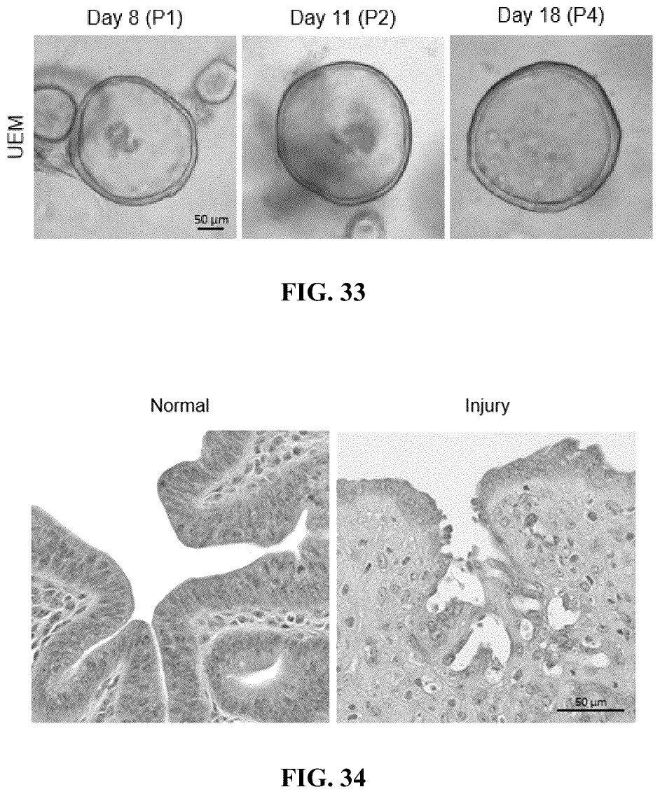

As shown in FIG. 33, endometrial organoids were passaged four times in 5 mg/mL UEM hydrogel (a total of 18 days of culture), and it was confirmed that the culture was successful.

Therefore, it was confirmed that long-term passaging of endometrial organoids using the UEM hydrogel was possible.

### Example 16: Establishment of Animal Model for In Vivo Transplantation of Endometrial Organoids Cultured Using Decellularized Uterine Tissue-Derived Hydrogel Scaffold

A 27G syringe needle was inserted under the fallopian tube of the mouse uterine horn and the endometrium was scraped 10 times, thereby establishing an endometrial damage mouse model in which fibrosis and adhesion induced in the endometrial layer were induced. The animal model was applied for transplantation of the endometrial organoids cultured in the UEM hydrogel (FIG. 34).

### Example 17: Evaluation of In Vivo Transplantation and Regeneration Effects of Endometrial Organoids Cultured Using Decellularized Uterine Tissue-Derived Hydrogel Scaffold

To check in vivo engraftment of organoids, organoids cultured for 4 days in Matrigel or 5 mg/mL UEM hydrogel were separated from the hydrogel and labeled with DiI, a fluorescent dye. The endometrial cells labeled with the fluorescent dye were mixed with Matrigel or UEM solution and injected into the submucosa of the uterus immediately after damage to the mouse endometrium. More specifically, 1.0×10⁶ cells were transplanted per mouse, and 50 μL Matrigel in solution before crosslinking or 50 μL UEM solution (adjusted to pH 7 by adding NaOH) was used.

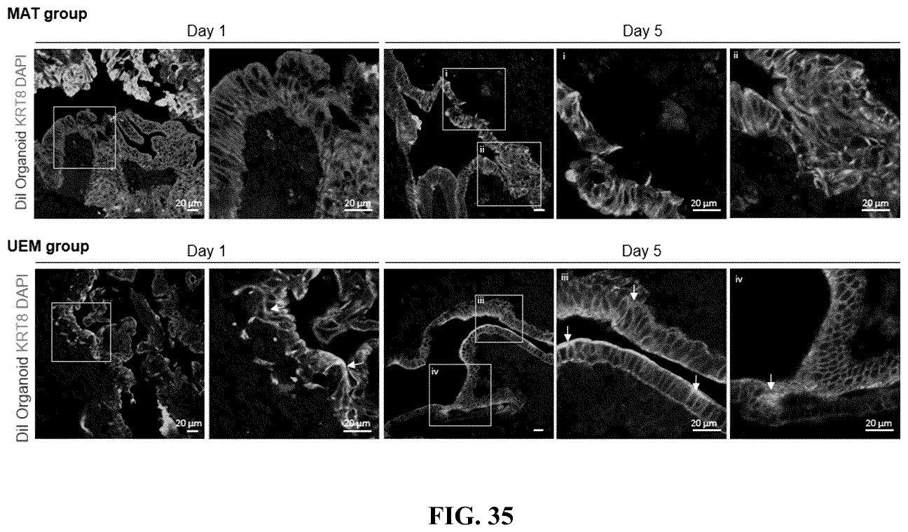

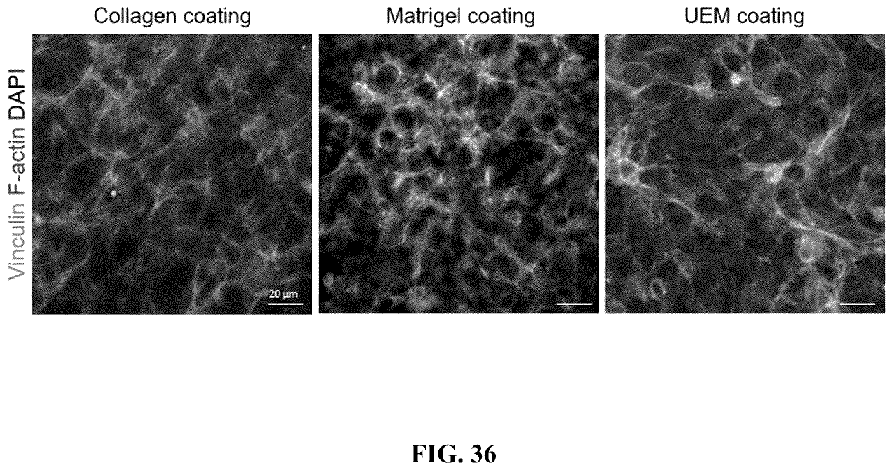

On days 1 and 5 after transplantation, damaged tissue was collected and engraftment of the DiI-labeled endometrial organoids was checked. It was confirmed that more endometrial organoids were located at and near the damaged endometrial site in the group transplanted with organoids cultured using the UEM hydrogel than in the group transplanted with organoids cultured using Matrigel, and that the transplanted organoids stably expressed cytokeratin 8 (KRT8), an epithelial tissue marker (DiI+KRT8+co-positive cells=indicated by white arrows) (FIG. 35).

This suggests that the UEM hydrogel can be used a scaffold support not only for culture of endometrial organoids but also for efficient in vivo transplantation of organoids.

### Example 18: Verification of Applicability of Decellularized Uterine Tissue-Derived Extracellular Matrix Composition as Coating Material

To verify the applicability of the decellularized uterine tissue-derived extracellular matrix composition as a culture surface coating material, a culture dish and Transwell were coated with each of 50 μg/mL UEM, 20 μg/mL type 1 collagen, 2% (v/v) Matrigel and applied for culture of endometrial organoids.

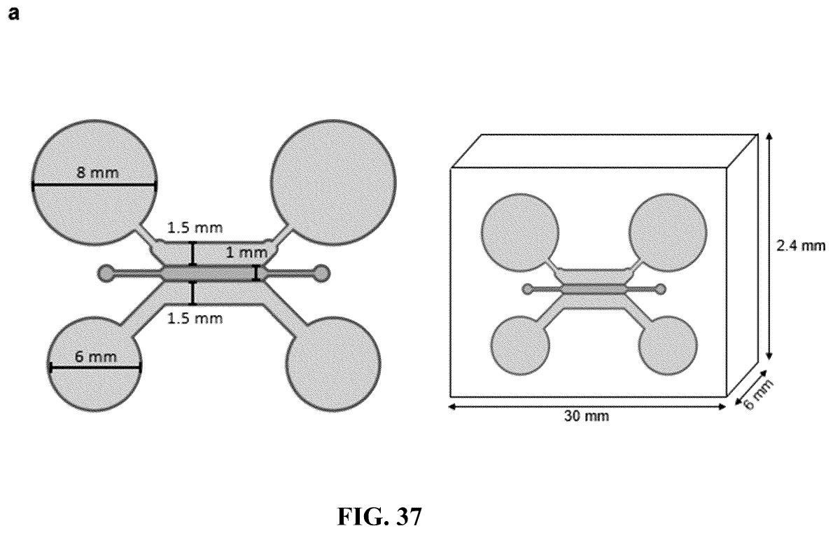

Endometrial organoids were dissociated into single cells, and then cultured on the UEM-coated culture dish and transwell for 4 days and analyzed by immunostaining. As a result, it was confirmed that the endometrial organoids formed a monolayer. Thereby, it was confirmed that a monolayer was formed in all of the UEM-, collagen-, and Matrigel-coated groups, and the degree of contact between cells was also similar between the groups (FIG. 36).

This suggests that the decellularized uterine tissue-derived (UEM) composition can be applied not only as a 3D hydrogel material for organoid culture but also as a coating material on the surface of a cell culture vessel, and enables attachment and culture of organoids, indicating that it can be used in a significantly expanded range of applications.

### Example 19: Fabrication of Endometrial Organoid Chip Using Decellularized Uterine Tissue-Derived Hydrogel Scaffold

For the purpose of developing an endometrial chip to mimic the function and morphology of the endometrium, a biomimetic endometrial chip was fabricated using endometrial organoids and UEM.

The endometrial chip made of polydimethylsiloxane (PDMS) consisted of a 1.5-mm-high upper medium and endometrial organoid culture channel, a 1-mm-high UEM scaffold layer middle channel, and a 1.5-mm-high lower medium channel. The overall size of the chip was 30 mm wide, 6 mm long, and 2.4 mm high (a in FIG. 37).

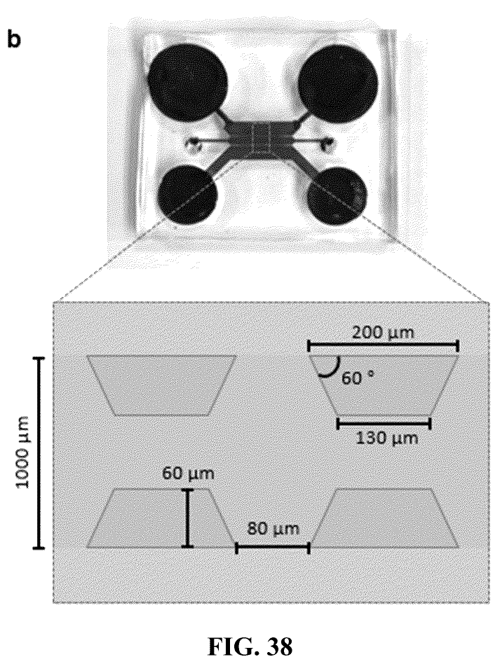

The UEM scaffold layer middle channel consisted of 16 trapezoidal posts arranged at intervals of 80 μm at each of the upper and lower portions. At this time, each post had a bottom side of 200 μm, an upper side of 130 μm, a height of 60 μm, and was manufactured to have an acute angle of 60° (b in FIG. 38).

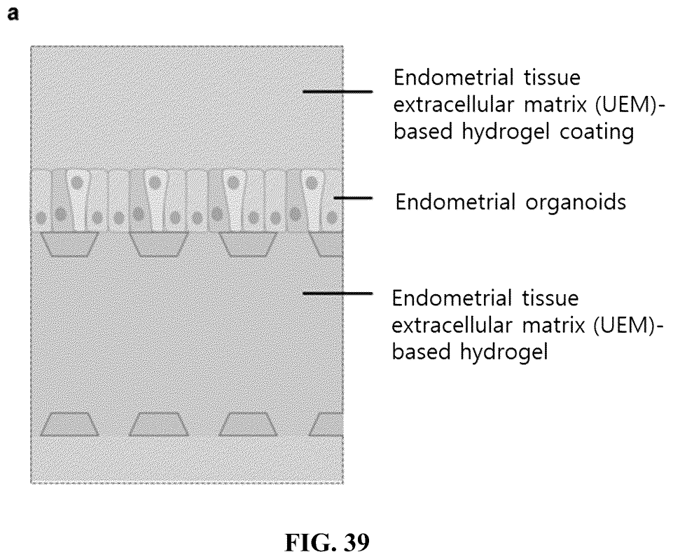

For stable culture and support of endometrial organoids, after coating with poly-L-lysine at a concentration of 20 μg/mL at 37° C. for 4 hours, 5 mg/mL UEM solution was injected into the middle channel layer and gelation thereof was induced at 37° C. for 4 hours. Thereafter, 5 mg/ml UEM solution was passed through the endometrial organoid culture channel to coat the inner wall of the channel with the UEM solution, and then gelated at 37° C. for 30 minutes. Finally, 50 g/mL UEM solution was injected, followed by coating at room temperature for 30 minutes (a in FIG. 39).

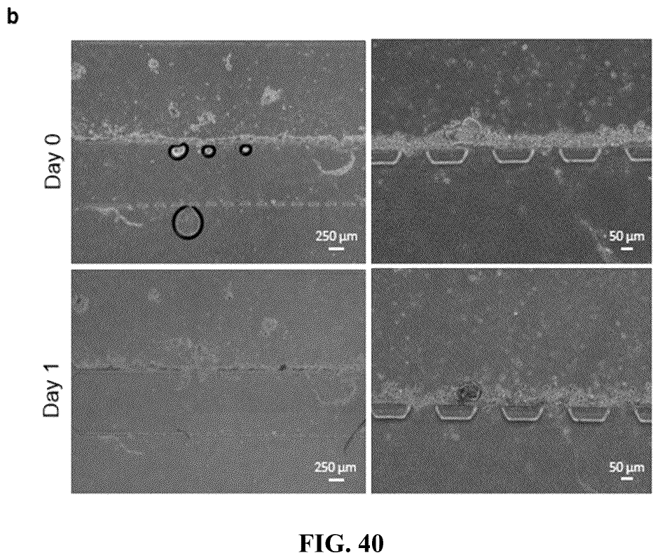

Endometrial organoids were dissociated into single cells by treatment with TrypLE containing 10 μM Y-27632 at 37° C. for 5 minutes, and then injected into the upper layer culture channel of the endometrial chip at a cell concentration of 2×10⁷ cells/mL. Immediately after cell injection, the endometrial chip was placed vertically and incubated so that the endometrial organoids could form a monolayer (b in FIG. 40).

In addition, a biomimetic endometrial chip of a different design was used to induce the formation of an endometrial monolayer.

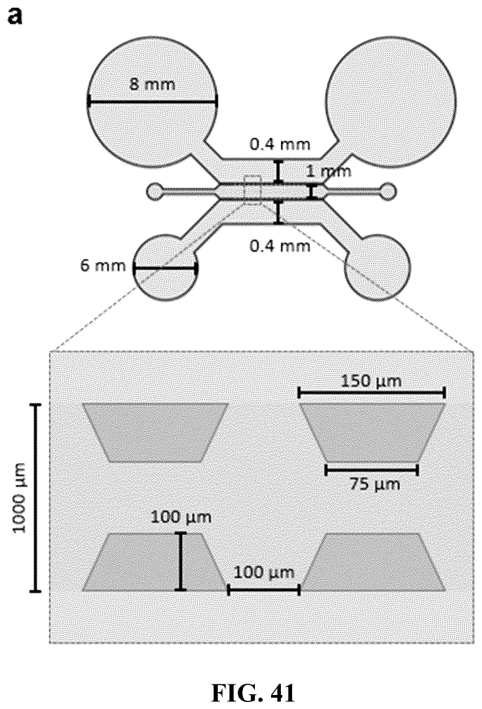

(a in FIG. 41) The endometrial chip consisted of a 0.4-mm-high upper layer medium and endometrial organoid culture channel, a 1-mm-high UEM scaffold layer middle channel, and a 0.4-mm lower layer medium channel. For stable culture and support of endometrial organoids, after coating with poly-L-lysine at a concentration of 20 μg/mL at 37° C. for 4 hours, 5 mg/mL UEM solution was injected into the middle channel layer and gelation thereof was induced at 37° C. for 30 minutes. Thereafter, 5 mg/mL UEM solution was passed through the endometrial organoid culture channel to coat the inner wall of the channel with the UEM solution, and then gelated at 37° C. for 30 minutes. Finally, 50 μg/mL UEM solution was injected, followed by coating at room temperature for 30 minutes.

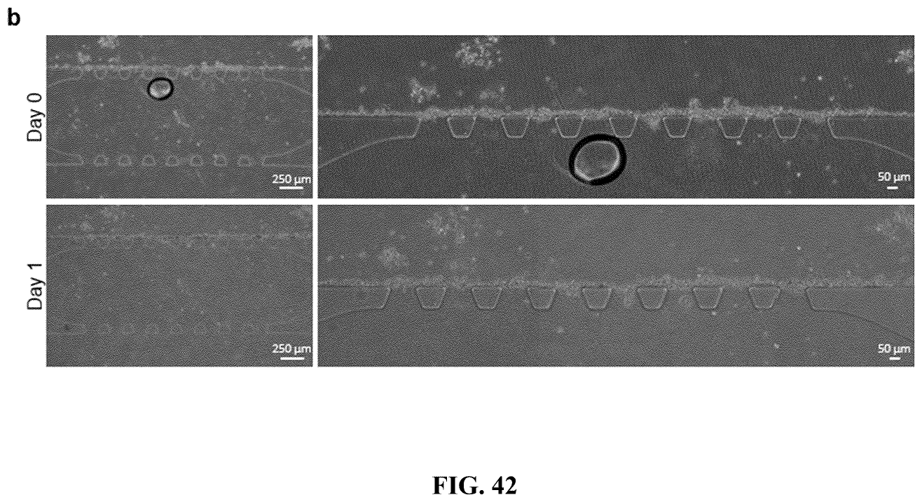

(b in FIG. 42) Endometrial organoids were dissociated into single cells by treatment with TrypLE containing 10 μM Y-27632 at 37° C. for 5 minutes, and then injected into the upper layer of the endometrial chip at a cell concentration of 2×10⁷ cells/mL. Immediately after cell injection, the endometrial chip was placed vertically and incubated so that the endometrial organoids could form a monolayer.

As a result, it was confirmed that the endometrial organoid chip could be fabricated using the decellularized uterine tissue-derived hydrogel scaffold.

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A hydrogel composition for three-dimensional culture of endometrial organoids, comprising decellularized uterine tissue-derived extracellular matrix (UEM).

2. The composition according to claim 1, wherein the decellularized uterine tissue-derived extracellular matrix contains COL6A3, COL6A1, or COL6A2 as collagen type VI, or FGA, FGB, or FGG as fibrinogen.

3. The composition according to claim 1, wherein the decellularized uterine tissue-derived extracellular matrix has a higher elastic modulus (G′) than a viscous modulus (G″) thereof.

4. The composition according to claim 1, wherein the decellularized uterine tissue-derived extracellular matrix has an elastic modulus of 10¹ to 10² Pa and a viscous modulus of 10⁰ to 10¹ Pa when a concentration of the decellularized uterine tissue-derived extracellular matrix is 1 to 8 mg/mL.

5. The composition according to claim 1, wherein a concentration of the decellularized uterine tissue-derived extracellular matrix is 1 to 8 mg/mL.

6. An endometrial organoid cultured in the hydrogel composition for three-dimensional culture according to claim 1.

7. The endometrial organoid according to claim 6, wherein the endometrial organoid expresses ERα, E-cadherin, or PanCK as a protein.

8. The endometrial organoid according to claim 6, wherein the endometrial organoid expresses Esr1, Lgr5, Foxa2, or Muc1 as a gene.

9. The endometrial organoid according to claim 6, wherein mucin secretion from the endometrial organoid is increased when the endometrial organoid is treated with estradiol or progesterone.

10. A method for preparing a hydrogel composition for three-dimensional culture of endometrial organoids, comprising steps of:
mixing uterine tissue with Triton X-100 and ammonium hydroxide;
preparing decellularized uterine tissue-derived tissue extracellular matrix by freeze-drying and grinding the tissue;
solubilizing the decellularized uterine tissue-derived extracellular matrix in a pepsin solution; and
mixing the solubilized extracellular matrix with PBS buffer, distilled water, and NaOH, followed by gelation.

11. The method according to claim 10, wherein the step of solubilizing the decellularized uterine tissue-derived extracellular matrix in the pepsin solution is performed at a temperature of 15 to 30° C.

12. The method according to claim 10, wherein a concentration of the pepsin solution in the step of solubilizing the decellularized uterine tissue-derived extracellular matrix in the pepsin solution is 3 to 10 mg/mL.

13. A method for producing endometrial organoids, comprising a step of culturing endometrial organoids in the hydrogel composition for three-dimensional culture of endometrial organoids according to claim 1.

14. The method according to claim 13, further comprising a step of passaging the endometrial organoids in WNT3a conditioned medium (CM).
